(19) United States
(12) Patent Application Publication
(10) Pub. No.: US 2007/0154536 A1
(43) Pub. Date: Jul. 5, 2007

(54) Drug delivery method by enhanced topical application

(75) Inventor: Chryslain Sumian, Jena (DE)

Correspondence Address: Dr. Bolesh J. Skutnik;CeramOptec Industries, Inc, 515 Shaker Road, East Longmeadow, MA, 01028, US

(73) Assignee: CeramOptec Industries Inc.

(21) Appl. No.: 11/706,720

(22) Filed: Feb. 15, 2007

Related U.S. Application Data
(63) Continuation-in-part of application No. 09/621,146, filed on Jul. 21, 2000.

(51) Int. Cl. A61K 9/70

(52) U.S. Cl. 424/449

(57) ABSTRACT

Methods for enhancing penetration of compounds/drugs into an animal or human through their hair follicles are provided. The methods employ topical application of swellable compositions which can maintain a passage for desired drugs by either opening hair follicles and preventing them from collapsing or increasing the depth of inner lumen space of the hair follicles. The swellable composition can be polymers that are biodegradable, bioactive, encapsulated in microspheres or liposomes, and/or form microspheres. The method is useful to increase compound/drug penetration deep into a patient, to increase flux of compounds/drugs through the hair follicle, to obtain release of compounds/drugs in the tissues surrounding hair follicle or under the skin surface, and to obtain a systemic effect of the compounds/drugs after topical application. The method is also useful to increase therapeutic effects of compounds/drugs in the treatment of a wide variety of skin disorders. The method can further used to obtain permanent removing of unwanted hair by permitting the diffusion of compounds/drugs deeply into hair follicles.

### REFERENCE TO RELATED CASE

This application is a continuation-in-part of co-pending U.S. patent application Ser. No. 09/621,146 filed on Jul. 21, 2000 by Chryslain Sumian, inventor, entitled “ENHANCING COMPOUND PENETRATION INTO AND THROUGH HAIR FOLLICLES”, and incorporated by reference herein.

### BACKGROUND OF THE INVENTION

1. Field of the invention

The present invention relates generally to the fields of dermatology and cosmetology and in particular relates to topical drug/compound delivery systems.

2. Invention Disclosure Statement

An average person weighing 65 Kg has approximately 13,000 cm² of exposed skin surface area, and this surface area is increased by about 30% because of fine wrinkles. The primary function of skin is to provide protections against abrasions and microorganisms, as well as to reduce water loss. In general, the skin acts as a barrier with permeability to the environment. This barrier function is achieved by a specific 4-layer assembly. The first layer is stratum corneum, which is between 10 to 20 μm thick. Underlying this region is epidermis (50-100 μm thick), dermis (1-2 mm), and hypodermis (1-2 mm) respectively. The primary barrier to external environment (i.e. outside the human body) is located within the superficial layer, the stratum corneum.

Topical drug delivery has become one of the most exciting and challenging areas of pharmaceutical research in the last decade because of its advantage over conventional drug therapy, such as muscle or blood vessel injection and oral administration. Topical drugs encompass both dermal and transdermal products, which are used for local and systemic effects.

Penetration enhancers have been examined for several years, and their significance has become greater with the development of transdermal drug delivery system. The term “enhancer” is used here to refer to agents or processes that decrease the barrier function of the skin. Reduction of skin barrier function increases the therapeutic efficacy of dermatological formulations and transdermal devices by obtaining significant improvements in the kinetics and/or extent of percutaneous absorption. The general requirements for enhancers and their formulations are similar to the requirements for a dermatological formulation, i.e. odorless, tasteless, cosmetically acceptable, chemically and physically stable, cost effective, and not provoking irritation or sensitization. Furthermore, the activity of such agents should be readily controlled. For example, the barrier function should be readily switched off upon application of an enhancer and subsequently restored after a compound is delivered and the preparation removed.

Because of their wide varieties of chemical structure, penetration enhancers act by more than one mechanism, and the precise enhancer activity depends on their physicochemical properties. Because of the skin structure (composition and function), there are only three possible routes to achieve percutaneous absorption: (1) transcellular, (2) intercellular, and (3) through the appendages, such as hair follicles and sweat glands. The relevance of these routes to percutaneous absorption of a compound depends on the area and the diffuseness and solubility of the compound in each domain. These pathways are not mutually exclusive. For example, water penetration through the stratum corneum is likely to follow all three pathways.

The hair follicles and sweat glands are sites of a natural, physical discontinuity in the stratum corneum and therefore have the advantages to serve as a penetration pathway. Appendages account for only 0.1-1% of the surface area of the skin and only 0.01-0.1% of the total skin volume. In order to significantly influence the flux of compounds across the skin (e.g. 10-fold) through the appendages, the diffusion has to be more than three orders of magnitude higher than that across the intercellular lipid domains or corneocytes. For this reason, it is likely that “shunt” pathways are relatively more important for molecules which exhibit relatively slow rates of percutaneous absorption and are of primary importance during early stages after topical application. Appendages, such as hair follicles, are relatively poor barriers to diffusion and consequently the lag time (i.e. time to reach steady state) is very short (minutes). However, the surface area of the follicular pathway is relatively small and consequently the maximum flux is correspondingly very slow (0.5-1 10⁻⁶ cm/h). In contrast to the shunt pathway, the diffuseness through the intercellular lipid domain is comparatively low and consequently there is a correspondingly longer lag time (hours). The relatively large surface area of this pathway contributes to a maximum flux corresponding to 10⁻³ cm/h for compounds with the appropriate physicochemical characteristics. Thus, the follicle pathway is more important at early time periods prior to the establishment of steady state of absorption through the intercellular lipid domain, and more important for compounds which demonstrate a relatively low diffusion rate through the stratum corneum.

Appendages may also serve as sites for depositing particles. Rolland A et al., Site-specific drug delivery to pilosebaceous structures using polymeric microspheres, Pharmaceutical Research 10: 1738-44 (1993) clearly demonstrated this by following the localization process of fluorescent microspheres to hair follicles. Small microspheres (<1 μm in diameter) enter in follicles as well as the upper 2-3 cellular layers of the stratum comeum and thus appear to be spread over the skin. In contrast, medium size microspheres (around 5 μm) enter in the follicles but are excluded from penetrating the upper layers of the stratum corneum. This results in an apparent targeting of these microspheres to hair follicles. Large microspheres (>10 μm) are excluded from penetrating into either of these sites. Consequently, appropriate choice of particle size facilitates specific follicular targeting. However, penetration of particles from the inner lumen of the follicles into surrounding living tissue has not been observed and particles penetrate to levels corresponding to 200-300 μm below the skin surface. Lieb LM et al., Topical delivery enhancement with multilamellar liposomes into pilosebaceous unit : I. In vitro evaluation using fluorescent techniques with the hamster ear model, J Invest Dermatol 99:108-113 (1992) and Hoffman RM, Topical liposome targeting of dyes, melanins, genes, and proteins selectively to hair follicles, J Drug Target, 5(2):67-74 (1998) show that the disposition of liposomes in hair follicles and pilosebaceous glands corroborate these observations. Thus, delivery of compounds from formulations containing particulate size of ingredients (microsphere or liposomes) may target compounds to follicles. Compounds may diffuse into the dermal tissue from the site of deposition upon dissolution or release. These results are obtained without hair removal.

As described above, most methods regarding to improvement of compound/drug penetration employ stratum corneum alterations/modifications. The activity of agents used in these methods should be controlled so that they enhance compound/drug penetration during application but do not affect restoration of the skin barrier function afterward. Nevertheless, this restoration of skin barrier function is not achieved in all present methods, and these methods induce potential side effects, i.e., irritation due to foreign compound penetration and dry skin.

Others approaches use appendages to enhance compound/drug penetration without skin barrier alteration. The principal limitations of these pathways are the low compound/drug flux through appendages and the lack of compound/drug penetration from appendages to dermis.

To increase flux, it's possible to remove hair from its hair follicle by using any known methods, such as cold wax, warm waxing, and mechanical devices to create a channel which leads directly and deeply into the follicle and greatly increases the ability of the follicle to take up agents. U.S. Pat. No. 5,989,267 discloses the increasing of compound/drug penetration by hair removal. However, structure of hair is harder than the tissue around it. When hair is removed, the pressure exerted by dermis (e.g. collagen and elastic fibers) produces follicle closing which is called follicle collapse. However, Sumian C et al., A new method to improve penetration depth of dyes into the follicular duct: Potential application for laser hair removal, J. Am. Acad. Dermatol., 41:172-5 (1999) report that dyes (e.g. Rhodamine 6G) can pass specifically through the follicle collapse if the dye was encapsulated in size-defined microspheres (around 5 μm in diameter) and diffusion outside the microspheres is induced. This diffusion can reach 500 μm below the skin surface (with the appropriate vehicle). After diffusion, compounds may stay in hair follicles to induce a specific action and/or diffuse into the dermal tissue. Penetration and diffusion of compounds/drugs inside hair follicles depend on formulation vehicle and molecule ability to pass through the follicle collapse (i.e. size, molecular weight, and solubility). Even if diffusion occurs, the compound/drug flux is limited by follicle collapse.

For the foregoing reasons, there is a need to develop new methods, which are efficient, easily administered, non-irritating, and capable of opening hair follicles to enhance compound/drug diffusion deeply along the hair follicles.

### Brief SUMMARY AND OBJECTS OF THE INVENTION

It is an object of the present invention to provide methods to enhance entry of topical compositions to increase compound/drug penetration in the skin without stratum corneum modification, or with skin barrier restoration after treatment.

It is a further object of the present invention to provide methods for systemic distribution of compounds/drugs by means of topical application of compositions containing such materials.

It is another object of the present invention to provide methods to enhance transfer for topical compositions across the natural skin barrier to benefit treatment in different hair disorders.

Yet another object of the present invention is to provide methods to enhance penetration of topical compound/drug compositions before treatment with radiation, and enhance compound/drug diffusion into the dermis.

Briefly stated, the present invention provides methods for enhancing penetration of compounds/drugs into hair follicles of an animal or human. The methods employ topical application of swellable compositions which can maintain a passage for desired compounds/drugs by either opening hair follicles and preventing them from collapsing, or increasing the depth of inner lumen space of the hair follicles. The swellable composition can be polymers that are biodegradable, bioactive, encapsulated in microspheres or liposomes, and/or form microspheres. The method is useful to increase compound/drug penetration deep into a hair follicle, to increase flux of compounds/drugs through the hair follicle, to obtain release of compounds/drugs in the tissues surrounding hair follicle or under the skin surface, and to obtain systemic effect of the compounds/drugs after topical application. The method is also useful to increase therapeutic effects of compounds/drugs in the treatment of a wide variety of skin disorders. The method is further used to obtain permanent removing of unwanted hair by permitting the diffusion of compounds/drugs deeply into hair follicle.

The above, and other objects, features and advantages of the present invention will become apparent from the following description read in conjunction with the accompanying drawings.

### BRIEF DESCRIPTION OF FIGURES

FIG. 1 shows the structure of the skin.

FIG. 2A-FIG. 2F illustrate the preferred embodiment in example 1.

FIG. 2A demonstrates how forces are applied to keep hair follicle collapsed.

FIG. 2B shows an enlarged view of a hair follicle after topical application of swellable composition.

FIG. 2C shows the hair follicle reaction after composition volume swelling is induced.

FIG. 2D illustrates enhanced penetration of topically applied compounds/drugs.

FIG. 2E shows the step of skin barrier restoration.

FIG. 2F shows the diffusion of the compounds/drugs either inside hair follicles or into other parts of the body.

FIG. 3A-FIG. 3G demonstrate the preferred embodiment in example 2.

FIG. 3A shows an enlarged view of a hair follicle after topical application of swellable composition.

FIG. 3B illustrates the hair follicle reaction after composition swelling is induced.

FIG. 3C shows the same reaction as FIG. 3B, but in an enlarged view of inner lumen of the hair follicle.

FIG. 3D shows topical application of compounds/drugs.

FIG. 3E demonstrates the same process as FIG. 3D but in an enlarged view of inner lumen of the hair follicle.

FIG. 3F shows the removal of the swelled composition.

FIG. 3G illustrates the diffusion of the compounds/drugs either inside hair follicles or into other parts of the body.

### DETAILED DESCRIPTION OF PREFERRED EMBODIMENTS

FIG. 1 shows the structure of the skin. The skin comprises 4 principal layers. The superficial region of the skin (first layer) is stratum corneum 1. Underlying this region is epidermis 2, dermis 3, and hypodermis 4. FIG. 1 also shows the structure of a pilosebaceous unit. The pilosebaceous unit is composed by hair follicle 5 and sebaceous gland 6. Each hair follicle 5 is a discontinuity in stratum corneum 1. The hair growing begins in hair bulb 7, located at the root of hair follicle 5. Hair follicle 5 is a tubular structure composed with five concentric layers of epithelial cells. In hair growth stage, epithelial cells proliferate to form the four internal hair follicle layers. During the progression from hair bulb to skin surface, three layers (in the center) are subjected to keratinization to form hair 8. The fourth layer disappears at the sebaceous gland level to leave a space, which is named inner lumen 9.

The present invention is further illustrated by the following examples, but is not limited thereby.

### EXAMPLE 1

A method for enhancing compound/drug penetration into hair follicles on the body areas of an animal or a human, wherein a composition is topically applied to cause the opening of hair follicles and prevent the follicles from collapsing. The following steps characterise this method:
(1) a preliminary epilation is performed, since hair is generally present in the body areas to which composition will be applied,.
(2) topical application of a swellable composition to said body areas with stratum corneum intact where compound/drug penetration enhancement is desired and where there are hair follicles.
(3) volume swelling of the composition applied in step (2) to prevent closure of said hair follicle and to enlarge where possible said depth of said hair follicle; and
(4) application of a cosmetically and/or pharmaceutically acceptable compound/drug

The term “epilation” as defined herein means that hair is removed from its hair follicle. Epilation can be performed using methods such as cold waxing, warm waxing, and the use of mechanical devices.

As used herein “topical application” means directly laying on or spreading on the skin of a mammal. This application could be performed with massaging. After this application, a substance, a film, a dressing and the like can be applied to achieve an occlusion.

The term “cosmetically and/or pharmaceutically acceptable composition” as defined herein can be salts, drugs, medicaments, inert ingredients or other materials which are suitable for use in contact with the tissues of humans or other animals without inducing toxicity, incompatibility, instability, irritation, allergic response, and the like reactions, commensurate with a reasonable benefit/risk ratio.

The term “swellable composition” relates to composition containing specific kinds of substance which swells. Examples of swellable composition or chemical cross-linked structures are disclosed in U.S. Pat. No. 5,770,229, 5,236,965, 5,026,596, and 4,596,858 as well as in WO 99/31167 and WO 99/08868, the disclosures of which are hereby incorporated by reference. The composition is a form of ointment, cream, lotion, gel, spray, tonic, mousse, paste and the like. In one preferred embodiment, the substance is a form of microsphere or liposome. As disclosed in EP 03750520 and WO 98/48716, formulations containing specific size of ingredients (e.g. microspheres or liposomes) may target compounds to follicles. As used herein “target” means specific penetration into hair follicles. By using this particular form of substances, hair follicles open without composition presence under the skin surface.

As used herein “compounds/drugs” mean any molecules used in cosmetic and/or pharmaceutics fields, including all photosensitizer molecules, their derivatives, and their precursors used in photodynamic therapy.

As used herein “photosensitizer molecules” include hematoporphyrin, indocyanine green, microcyanine, clorin, chlorophyll, dyes, carbon, ALA (aminolevulinic acid), benzoporphyrin, protoporphyrin and their derivatives. The list of photosensitizer molecules given here is non-limiting and is only for exemplification.

As used herein “penetration enhancement” means increasing the quantity of compounds/drugs into hair follicles or increasing compound/drug flux through hair follicles.

The term “volume swelling” relates actions that increase physical volume occupied by the composition. This swelling is induced by any known processes. The choice of the process used is carried out by the substance incorporated into the composition. These processes include solvent evaporation, pressure changing, physiological reactions, thermal reactions with or without external power supply, reactions induced by radiation such as photochemical, photothermal reactions, chemical reactions with another compound like water, and physical process such as ultrasound or pressure. The list of processes given here is non-limiting and is given only for exemplification. Preferentially, a plate, a film, a dressing and the like are applied above the composition during swelling process to favor horizontal swelling against vertical swelling. After swelling process, compositions are permeable and permit compounds/drugs passing through or around it (if composition is in a form of microsphere or liposome).

Eventually, a skin barrier restoration can proceed after compound/drug application. As used herein “restoration” relates to closing up hair follicles after treatment. This is achieved by canceling forces exerted to hair follicles from composition swelling. This cancellation is obtained by any known means choosing according to composition used. It can be biodegradation, stripping, dissolution in situ, optical reactions, temperature changing, chemical reactions, physical processes, or physiological reactions. The list of processes given here is non-limiting and is given only for exemplification.

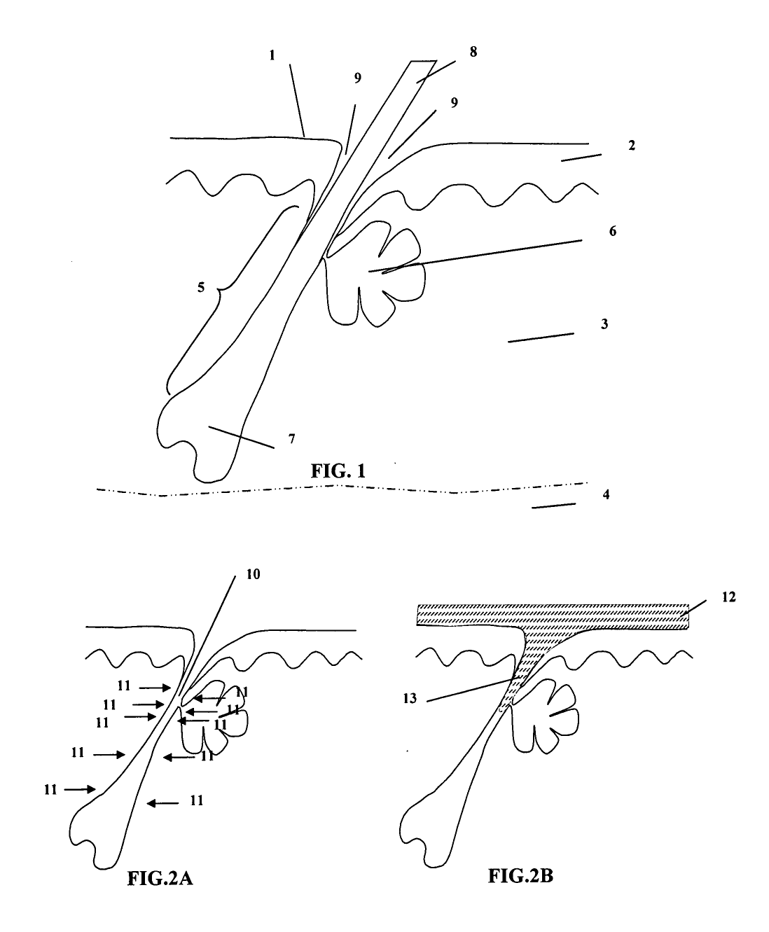

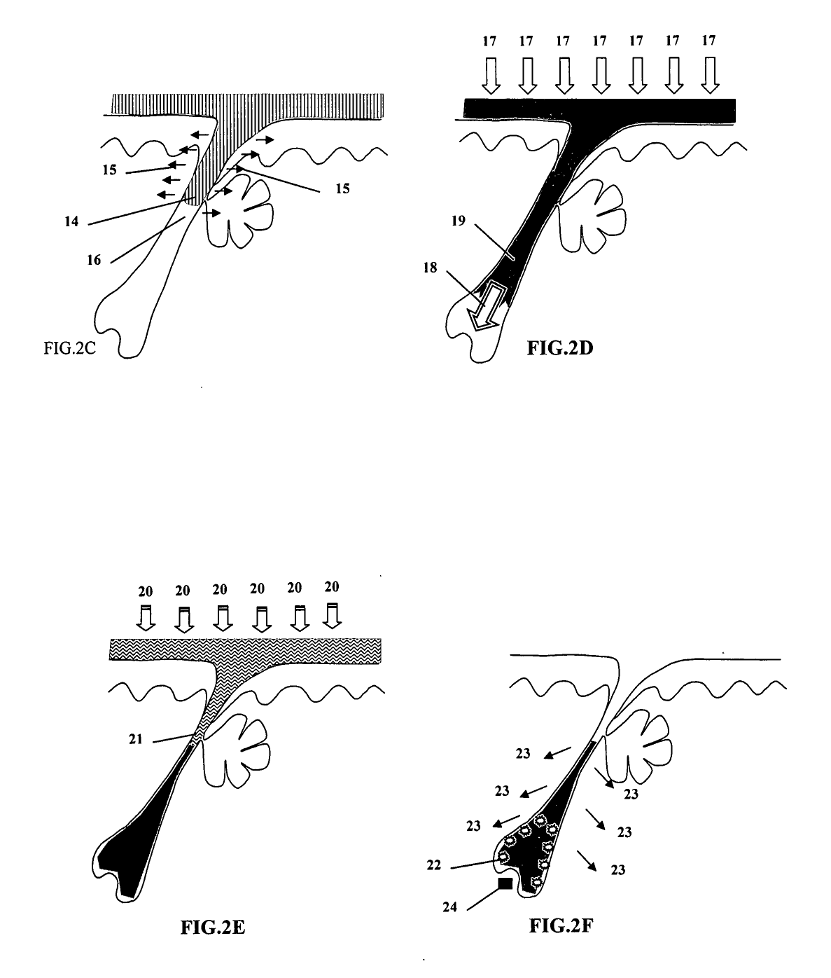

The dermis is composed of elastic and collagen fibers. These fibers provide the principal mechanical resistance of the skin and exert pressure on hair follicle. As it is depicted in FIG. 2A, hair follicle collapse 10 appear when hair is removed or when there are no “normal” growing (such as in alopecia). However, the difference on the structure along the hair follicle produce different mechanical reactions 11 and hair follicle collapses 10 only on the superficial part. Thus, compound/drug enhancement could be obtained by exerting strengths against the collapse for opening hair follicle.

FIG. 2B shows an enlarged view of a hair follicle after topical application of cosmetically and/or pharmaceutically acceptable and swellable composition 12. After topical application with light massaging, composition penetrates into the hair follicle down to collapsing position 13.

FIG. 2C shows the hair follicle reaction after inducing composition volume swelling. Swelling forces 15 exert pressure on hair follicle against the collapse. The result of this step is the hair follicle opening.

FIG. 2D shows topical application of compound/drug solution 17. Because of the permeability of swelled composition, compounds/drugs 17 pass through the composition and penetrate the skin. If there are no stratum corneum alterations, compounds/drugs 17 penetrate preferentially into hair follicle to increase flux 18 through it. This flux increasing induces even deeper penetration 19.

FIG. 2E shows the step of skin restoration. To prevent penetration of unwanted foreign compound after the treatment, skin barrier is restored by cancellation of the forces exerted by swelled composition. This step is accomplished by either a single or a combination of different methods, such as stripping composition, composition solubilization, and composition biodegradation 20. As used herein generally “skin barrier restoration” means hair follicle 21 collapse.

As shown in FIG. 2F, the compounds/drugs can diffuse inside and/or outside hair follicle 22 after the penetration. As used herein “outside diffusion” means into dermis 23 and/or into the blood by systemic passage 24.

### EXAMPLE 2

A method for enhancing compound/drug penetration into hair follicles on body areas of an animal or human is provided wherein a composition is topically applied and therefore causes depth of the inner lumen space of hair follicles to increase. The method comprises the steps of:
(1) Topical application of cosmetically and/or pharmaceutically acceptable and swellable composition on the skin areas where compound/drug penetration enhancement is desired and where there are hair follicles; and
(2) Volume swelling of the composition applied in step (1)
   Before the first step, hair on body areas where composition is going to be applied should be clipped.

The term “clip” as defined herein means that hair is cut. The cutting is performed by using different methods, such as a manual or electrical razor, a pair of scissors, and manual or electrical clippers.

As used herein “topical application,” “cosmetically and/or pharmaceutically acceptable composition,” “swellable composition,” “compound/drug,” “photosensitizer molecules,” “penetration enhancement,” “volume swelling” are the same as defined in example 1.

The swellable composition may target drugs to inner lumen of hair follicles. By using a particular form of composition, inner lumen is opened without composition presence under the skin surface.

Eventually, a skin barrier restoration can proceed after compound/drug application. As used herein “restoration” relates to cancel forces exerted to inner lumen of hair follicles by composition swelling. This cancellation is obtained by any known means choosen according to the composition used. It can be biodegradation, stripping, dissolution in situ, optical reaction, temperature changing, chemical reaction, physical process, and physiological reaction. The list of processes given here is non-limiting and is given only for exemplification.

In this example, there is no hair removal and no hair follicle collapse. The fourth layer of the hair follicle provides a junction between external and internal layers to prevent any foreign compound penetration. By exerting forces at the inner lumen of hair follicle, the space is enlarged because of the alteration of the layer, and compounds/drugs can diffuse through the layer.

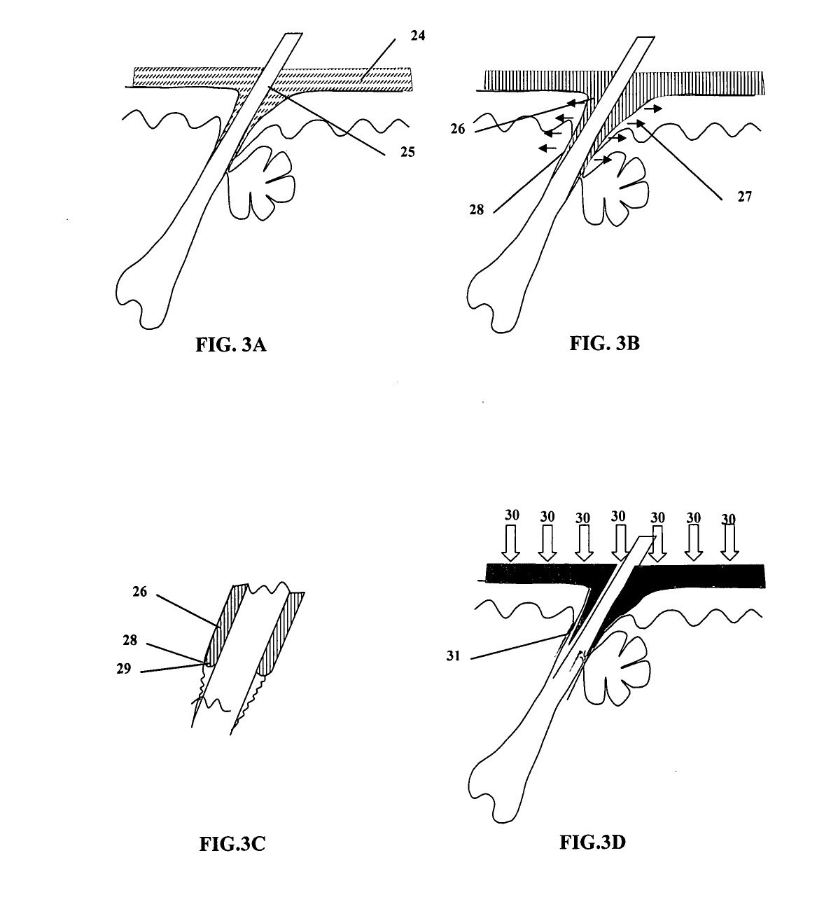

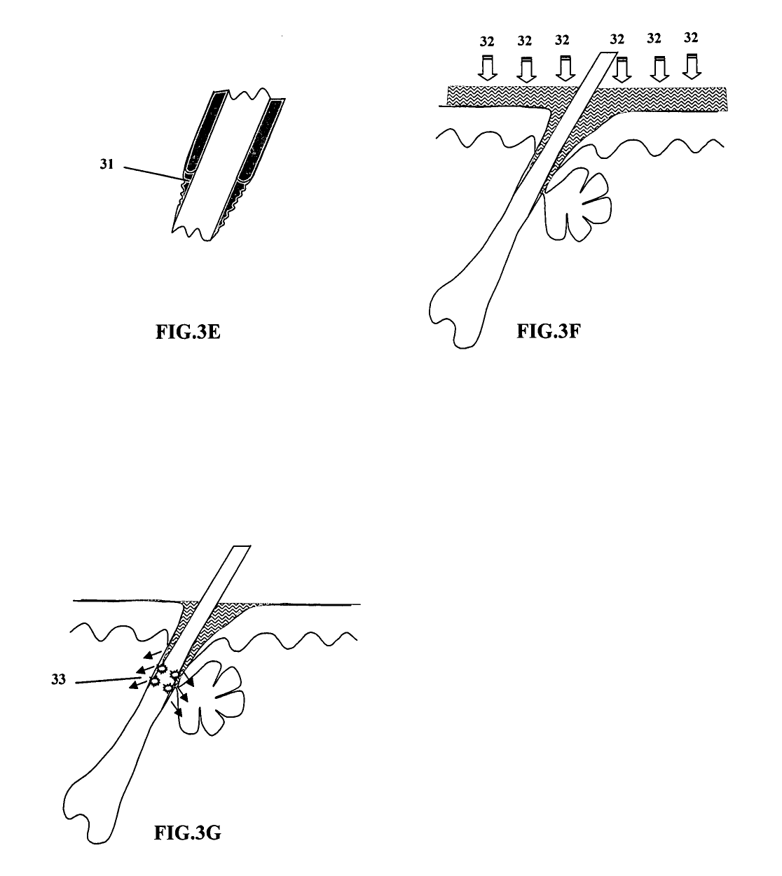

FIG. 3A shows an enlarged view of a hair follicle after topical application of cosmetically and/or pharmaceutically acceptable and swellable composition 24. After topical application with a light massaging, composition penetrates into the inner lumen 25 of the hair follicle.

FIG. 3B and FIG. 3C (enlarged view of inner lumen) show hair follicle reaction after inducing swelling of composition 26. Forces 27 created by swelling exert pressure on external layer 28 of the hair follicle. The result of this step is alteration of the junction between fourth 28 and third 29 hair follicle layers.

FIG. 3D and FIG. 3E (enlarged view of inner lumen) show topical application of compound/drug solution 30. Because of permeability of swelled composition, compounds/drugs 30 pass through the composition to penetrate the skin. If there are no stratum corneum alterations, compounds/drugs penetrate preferentially into hair follicle to increase flux 31 through it. This flux increasing induces an increasing of penetration depth.

FIG. 3F shows the removal of swelled composition. This step is accomplished by a single method or a combination of different methods, such as stripping composition, composition solubilization, and composition biodegradation 32.

FIG. 3G shows compounds/drugs can diffuse inside and/or outside hair follicle 33 after the penetration.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. A method for enhancing compound/drug penetration into an animal or human body through hair follicles on their body areas, comprising the steps of:
a. a pretreatment step of removing hair from said hair follicles;
b. applying topically a swellable composition to said body areas with stratum corneum;
c. insuring entry of said swellable composition into said hair follicle lumen;
d. preventing closure of said hair follicle lumen by volume swelling of said swellable composition in said lumen, wherein internal forces are generated by said swellable composition at said hair follicles during and after said volume swelling of said composition to open said hair follicles and prevent collapse of said follicle lumen; and
e. applying a compound/drug to said hair follicle area through said follicle lumen for increased application of said compound/drug to animals and humans.

2. The method according to claim 1, wherein said swellable composition includes an active compound/drug.

3. The method according to claim 1, wherein said swelling enlarges a depth of an inner lumen space of said hair follicles for increasing a surface area for enhancing the absorption of said compound/drug into said body areas.

4. The method according to claim 1, further comprising another step after step d: occluding said hair follicles.

5. The method according to claim 1, wherein said compound/drug are photosensitizer molecules.

6. The method according to claim 1, wherein said swellable composition is composed of polymers.

7. The method according to claim 6, wherein said polymers are biodegradable.

8. The method according to claim 6, wherein said polymers are biologically active.

9. The method according to claim 6, wherein said polymers are encapsulated.

10. The method according to claim 6, wherein said polymers are encapsulated in liposomes.

11. The method according to claim 1, wherein said swelling composition is covered during said volume swelling step, and whereby horizontal swelling of said swelling composition is favored against vertical swelling out of said hair follicle.

12. The method according to claim 11, wherein said swelling composition is covered by a cover selected from a group consisting of a plate, a film and a dressing.

13. The method according to claim 1 used to deliver a compound/drug to a patient's dermis and through their blood systemically, through a topical application of said compound/drug to body areas with stratum comeum.

14. The method according to claim 13, which is a pretreatment to a photodynamic therapy treatment which occurs away from said topical application site.

15. A method to achieve systemic introduction of compounds/drugs without using needles, needle patches, or oral means.

16. The method according to claim 15, wherein said compounds/drugs are photosensitizer molecules and their precursors, used in photodynamic therapy.
